(19) United States
(12) Plant Patent Application Publication
(10) Pub. No.: US 2001/0011383 P1
(43) Pub. Date: Aug. 2, 2001

(54) Chrysanthemum named Catchup Dark

(76) Inventor: Robert Noodelijk, Woubrugge (NL)

Correspondence Address: PARKHURST & WENDEL, L.L.P., SUITE 210, 1421 PRINCE STREET, ALEXANDRIA, VA, 22314-2805, US

(21) Appl. No.: 09/788,456

(22) Filed: Feb. 21, 2001

(30) Foreign Application Priority Data
Aug. 8, 1997 (NL) CHR2967
Jul. 15, 1998 (EP) 1998/0971

(51) Int. Cl. A01H 5/00

(52) U.S. Cl. PLT/298

(57) ABSTRACT

A Chrysanthemum plant named Catchup Dark characterized by its small sized blooms with red ray-florets and yellow-green disc florets.

### RELATED APPLICATIONS

The present application is related to the following co-pending applications: Application no. Filing Date Title

### BACKGROUND OF THE INVENTION

The new plant of the present invention comprises a new and distinct cultivar of chrysanthemum plant that is a natural occuring sport of a parent chrysanthemum named Catchup. A comparison with Parent chrysanthemum Catchup is also made in this application. The new cultivar was discovered as a whole plant mutation in May 1997 by Rob Noodelijk in a controlled environment (greenhouse) in Rijsenhout Holland. The first act of asexual reproduction of Catchup Dark was accomplished when vegetative cuttings were taken from the initial selection in July 1997 in Rijsenhout Holland.

### SUMMARY OF THE INVENTION

The present invention is a new and distinct variety of chrysanthemum bearing small sized blooms with red ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a stem of the cultivar in full bloom.

FIG. 2 shows the various stages of bloom and foliage of the new cultivar.

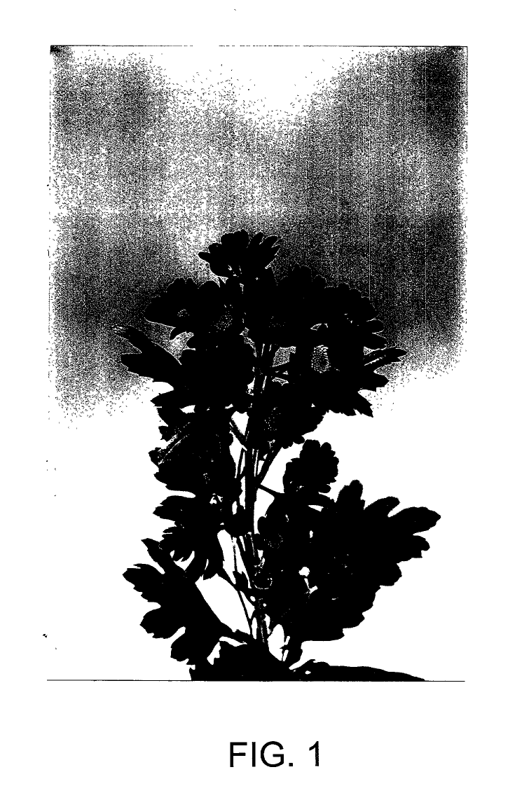

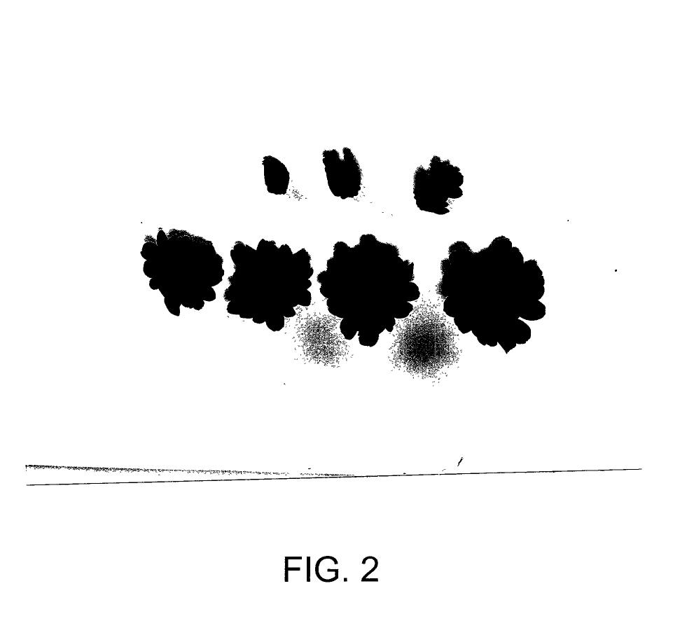

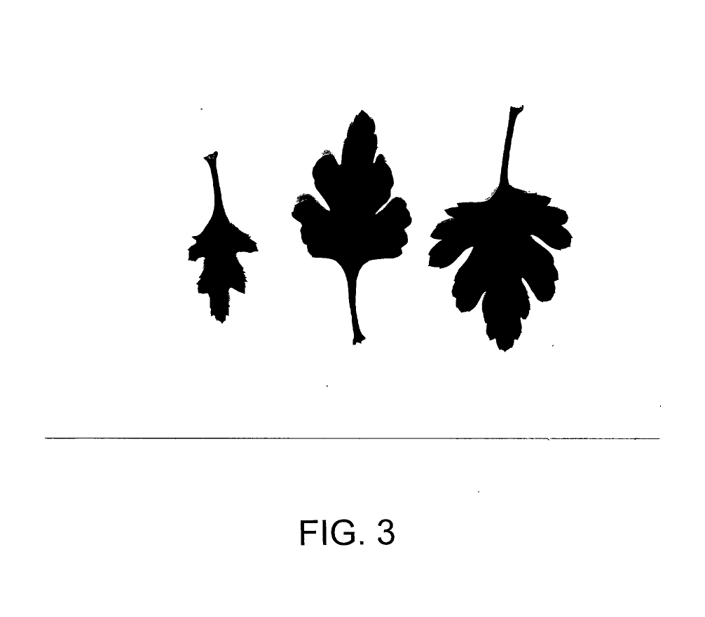

### DESCRIPTION OF THE INVENTION

This new variety of chrysanthemum is of the botanical classification Dendranthema grandiflora. The observations and measurements were gathered from plants grown in a greenhouse in Rijsenhout Holland in a photo-periodic controlled crop. The photo-periodic response time in this crop was 49 days. This new variety produces small sized blooms with red ray-florets and yellow-green disc-florets blooming on the plant for 4 weeks. This new variety of chrysanthemum has been found to retain its distinctive characteristics throughout successive propagations however the phenotype may vary significantly with variations in environment such as light intensity and temperature. To show the phenotype as described Catchup Dark may be planted without assimilation lightning between week 50 and 40 under greenhouse conditions in Holland. With assimilation lightning (minimum level 2500 lux) it may be planted year round under greenhouse conditions in Holland.

When Catchup and Catchup Dark are being compared the following differences and similarities are noticed: The difference of Catchup and Catchup Dark is the ray-floret color for which characteristics Catchup Dark has been selected out of Catchup. The ray-florets of Catchup Dark are darker red than the ray-floret color of Catchup. All other characteristics of Catchup and Catchup Dark are similar.

The following is a description of the plant and characteristics that distinguish Catchup Dark as a new and distinct variety. The color designations are taken from the plant itself. Accordingly, any discrepancies between the color designations and the colors depicted in the photographs are due to photographic tolerances.

| Botanical Description of | | |
|---|---|---|
| CULTIVAR Catchup Dark | | Catchup |
| Bud | | |
| Size | Cross-section +/− | |
| | 1.0 cm height +/− | |
| | 1.2 cm | |
| Outside Color | Greyed-purple | |
| | 187 A | |
| Bloom | | |
| Size | Small | |
| Fully Expanded | 3.5 cm | |
| Number of blooms per single stem | Average of 12 | |
| Peduncle length | Near the top +/− | |
| | 4 cm, near the | |
| | middle +/− 10 cm, | |
| | near the bottom +/− | |
| | 14 cm | |
| Peduncle lateral shoot attachment | Medium | |
| Peduncle lateral shoot angle | 30° | |
| Form | Single (daisy) | |
| Performance (blooming period) | 28 days | |
| Color | | |
| Center of the flower (disc-florets) | Immature yellow- | |
| | green 144A mature | |
| | yellow-green 144B | |
| Color of upper surface of the majo- | Greyed-purple 187A | Red 46A |
| rity of the ray-florets | | |
| Color of the lower surface of the | Greyed-purple 187B | Greyed- |
| majority of the ray-florets | | yellow 162D |
| Tonality from Distance | A small dark red | |
| | daisy | |
| Discoloration | None | |
| Ray florets | | |
| Texture | Upper and under | |
| | side smooth | |
| Number | 22-24 | |
| Cross-section | Concave | |
| Longitudinal axis of majority | Straight | |
| Shape of tip | Dentate | |
| Fragrance | Typical | |
| | chrysanthemum | |
| Disc Diameter (when visible) | Small to medium | |
| | (1.0-1.2 cm) | |
| Reproductive Organs | | |
| Stamen (present in disc florets only) | Yellow, thin, 3 mm | |
| | in length | |
| Pollen | Not present | |
| Styles (present in both ray and disc | Green, thin | |
| florets) | | |
| Style Length | +/− 4 mm | |
| Stigmas | Green | |
| Stigma Width | +/− 1 mm | |
| Ovaries | Enclosed in calyx | |
| Plant | | |
| Form | A spray mum meant | |
| | for indoor use | |
| Growth | Medium | |
| Height | 100 cm | |
| Stem Color | Yellow-green 146C | |
| Stem Strength | Medium | |
| Stem Brittleness | Absent | |
| Stem Anthocyanin Coloration | Present | |
| Flowering Response(photo-periodic | 49 days | |
| controlled crop, no natural growing) | | |
| Foliage | | |
| Color | Upper side | |
| | green 137A | |
| | under side | |
| | green 137C | |
| Size | Length +/− 7 cm, | |
| | width +/− 5.5 cm | |
| Quantity (number per single stem) | 24-26 | |
| Shape | Egg-shaped | |
| Texture | Fleshy | |
| Ribs and Veins | Ribs and veins well | |
| | developed | |
| Edge | Crenated | |
| Shape of Base of Sinus Between | Acute | |
| Lateral Lobes | | |
| Margin of Sinus Between Lateral | Parallel to diverging | |
| Lobes | | |
| Shape of Base | Truncate | |
| Apex | Mucronate | |

I claim:

1. A new and distinct variety of chrysanthemum plant as described and illustrated.
